(19) United States
(12) Patent Application Publication
(10) Pub. No.: US 2001/0004522 A1
(43) Pub. Date: Jun. 21, 2001

(54) KINASE ACTIVITY MEASUREMENT USING FLUORESCENCE POLARIZATION

(76) Inventors: THOMAS J. BURKE, MADISON, WI (US); RANDALL E. BOLGER, OREGON, WI (US); REBECCA SCHALL, MADISON, WI (US); GREGORY PARKER, MADISON, WI (US)

Correspondence Address: MARK K JOHNSON, P O BOX 510644, NEW BERLIN, WI, 531310644

(21) Appl. No.: 08/959,367

(22) Filed: Oct. 28, 1997

(51) Int. Cl. C12M 1/34; C12M 3/00; G01N 33/53

(52) U.S. Cl. 435/4; 435/7.1; 435/287.1; 435/194

(57) ABSTRACT

A method for quantitating enzyme activity of phosphorylation and dephosphorylation of a peptide or protein substrate, comprising measuring the fluorescence polarization of a fluorescence-emitting reporter molecule in solution with the phosphate. Then, adding an enzyme, either a kinase or a phosphatase, and incubating the solution. Finally, measuring the fluorescence polarization of the solution after the enzyme has had an opportunity to react with the peptide or protein substrate.

### CROSS-REFERENCE TO RELATED APPLICATIONS

This is a Provisional Application Ser. No. 60/029,831, filing date Oct. 28, 1996.

### FEDERALLY SPONSORED RESEARCH

Not applicable

### FIELD

The field of the invention is the detection of phosphorylated amino acids using a fluorescence polarization (anisotropy) competition assay. In particular, the process of the invention detects and measures protein kinase activities and to monitor phosphatases. In another application, the assay can be used to quantitatively measure phosphorylated amino acids in extracts.

### BACKGROUND OF THE INVENTION

Kinases are enzymes that catalyze the transfer of a phosphate molecule from a nucleotide triphosphate to a substrate such as an amino acid. Typically the amino acids serine, threonine, histidine, and tyrosine are the phosphate acceptors in proteins and ATP is the phosphate donor. Kinases play a role in virtually all regulated cell pathways, from ion transport to metabolic pathways to DNA replication to developmental differentiation. For example, phosphorylation of a protein may induce activation via a conformational change or it may initiate a cascade of molecular binding events.

Several human pathologies such as diabetes, cancer, and the allergic response have been directly linked to faulty kinase regulation in cells. A mutated or missing protein can change a regulated pathway to be constitutively ‘on’ or ‘off’, disrupting the steady state balance of biological control. Many of the extracellular receptors transmit information to the nucleus via pathways involving kinases. For example, when interleukins and cytokines bind to the surface of the cell, their effects are typically modulated via kinases. Epidermal growth factor receptor, nerve growth factor receptor, platelet derived growth factor receptor, and fibroblast growth factor receptor all involve signal transduction via kinase pathways. Because kinases play such a fundamental role in cellular control, they are often targets for the development of new therapeutics. New drugs are sought which can restore control to faulty regulatory systems and diminish the pathological effects.

Typically, kinase activity is measured using radioisotopes such as phosphorus-32 or phosphorus-33. Using radiation provides for very sensitive assays but creates biological hazards and expensive disposal costs. In a typical reaction, a radioactive phosphate from radioactive ATP is incorporated into the amino acid via the kinase reaction. The unincorporated radioactive ATP is removed with a filter binding separation. The radioactivity that remains bound to the filter is counted in a scintillation counter and is then used to calculate protein kinase activity.

Another radioactive method called the SPA (scintillation proximity assay) can also be used for measuring kinase activity. In this pseudo-homogeneous assay, the radioactive substrate is brought is close proximity to a molecule which amplifies the energy emission signal from the radioactive source. While this method is still radioactive and requires several molecules to be covalently labeled, it does not require the separation step of the filter binding assay.

Non-radioactive methods have been developed but most require separation or washing steps or some reaction components need to be immobilized. In a 96 well assay, the kinase substrate (peptide or protein) are normally bound to the plastic wells. The kinase reaction is then performed and a tagged antibody is used to detect the phosphorylated amino acid. A detection system is then used to detect the tagged primary antibody and amplify the signal using systems such as alkaline phosphatase or horseradish peroxidase. These assays can be sensitive but are often labor intensive, expensive, and enzyme kinetic information can be limited because of the immobilized component. Often these assays are semi-quantitative because it is difficult or impossible to measure the amount of immobilized substrate.

Time-resolved fluorescent assays have also been developed to measure kinase activity. In these assays, fluorophores with very long fluorescent lifetimes such as the lanthanide chelates are used because their fluorescence emission allows detection long after most other fluorophores have emitted the light. They also can be coupled with a receptor molecule to capture the energy released during emission. These coupled time-resolved fluorescent assays offer good sensitivity if a laser light source is used. A significant drawback is that two molecules are labeled in this method and the chemicals that are required are not widely available.

### SUMMARY OF THE INVENTION

We have developed a simpler, more direct method for measuring kinase activity using fluorescence polarization assays. Fluorescence polarization is a versatile laboratory technique for measuring equilibrium binding, nucleic acid hybridization, and enzymatic activity. Fluorescence polarization assays are homogeneous in that they do not require a separation step such as centrifugation, filtration, chromatography, precipitation or electrophoresis. Assays are done in real time, directly in solution and do not require an immobilized phase. Polarization values can be measured repeatedly and after the addition of reagents since measuring the polarization is rapid and does not destroy the sample. Generally, this technique can be used to measure polarization values of fluorophores from low picomolar to micromolar levels.

When a fluorescently labeled molecule is excited with plane polarized light, it emits light that has a degree of polarization that is inversely proportional to its molecular rotation. Large fluorescently labeled molecules remain relatively stationary during the excited state (4 nanoseconds in the case of fluorescein) and the polarization of the light remains relatively constant between excitation and emission. Small fluorescently-labeled molecules rotate rapidly during the excited state and the polarization changes significantly between excitation and emission. Therefore, small molecules have low polarization values and large molecules have high polarization values. For example, a fluorescein-labeled peptide has a relatively low polarization value but when the peptide is bound to a very large protein, it has a high polarization value.

Fluorescence polarization (P) is defined as:

$$P = \frac{Int \vert \vert - Int ⊥}{Int \vert \vert + Int ⊥}$$

Where Int || is the intensity of the emission light parallel to the excitation light plane and Int ⊥ is the intensity of the emission light perpendicular to the excitation light plane. P, being a ratio of light intensities, is a dimensionless number. The Beacon™ and Beacon 2000™ System used in these experiments expresses polarization in millipolarization units (1 Polarization Unit=1000 mP Units).

The relationship between molecular rotation and size is described by the Perrin equation and the reader is referred to Jolley, M. (Jour. Anal Tox. 5: 236-240, 1991) which gives a thorough explanation of this equation. Summarily, the Perrin equation states that polarization is directly proportional to the rotational relaxation time, the time that it takes a molecule to rotate through an angle of approximately 68.5 degrees. Rotational relaxation time is related to viscosity (η), absolute temperature (T), molecular volume (V), and the gas constant (R) by the following equation:

$$Rotational Relaxation Time = \frac{3 η V}{RT}$$

The rotational relaxation time is small (≈1 nanosecond) for small molecules (e.g. fluorescein) and large (≈100 nanoseconds) for large molecules (e.g. immunoglobulins) (Jolley, 1981). If viscosity and temperature are held constant, rotational relaxation time, and therefore polarization, are directly related to the molecular volume. Changes in molecular volume may be due to interactions with other molecules, dissociation, polymerization, degradation, hybridization, or conformational changes of the fluorescently labeled molecule. For example, fluorescence polarization has been used to measure enzymatic cleavage of large fluorescein labeled polymers by proteases, DNases, and RNases. It also has been used to measure equilibrium binding for protein/protein interactions, antibody/antigen binding, and protein/DNA binding. In this patent, we will show that fluorescence polarization is a simple and economical way to measure protein kinase activity.

A method for measuring the presence of a phosphorylated amino acid of a compound by competition, comprising: measuring the fluorescence polarization of a reporter complex comprising a first phosphorylated amino acid bound to a binding molecule; adding a substance containing a second phosphorylated amino acid to compete for the binding molecule; incubating the solution; measuring the fluorescence polarization of the solution during step c); and, comparing the fluorescence polarization measurements.

A method for measuring enzyme activity for attaching and cleaving a phosphate with a compound by competition, comprising: measuring the fluorescence polarization of a reporter molecule comprising a phosphorylated amino acid; adding an enzyme, incubating the solution; measuring the fluorescence polarization of the solution during step c); and, comparing the fluorescence polarization measurements.

A kit utilizing the method of claim 1 for measuring the amount of phosphorylated molecules in a mixture, comprising: instructions for utilizing fluorescence polarization to identify the amount of phosphorylated amino acids in a mixture, a receptacle containing a reporter molecule; and, a receptacle containing a binding protein.

A method is described for measuring enzyme activity of attaching and cleaving a phosphate with a compound, comprising: measuring the fluorescence polarization of a reporter molecule in solution with the phosphate. Then adding an enzyme which is a phosphatase or a kinase in the preferred embodiments. Incubating the solution for a time sufficient to allow enzyme activity and measuring the polarization. Finally, comparing the fluorescence polarization of the solution in the first step with the fluorescence polarization measurements in the last step.

A method for measuring enzyme activity of attaching and cleaving a phosphate with a peptide, comprising: incubating a first phosphate, the peptide, and an enzyme in solution. Then, adding a reporter molecule to the solution and measuring the fluorescence polarization of the reporter molecule. Incubating the solution for a time sufficient to allow for measurement of the fluorescence polarization and comparing the fluorescence polarization measurements.

A kit utilizing the method of claim 1 for measuring enzyme activity, comprising: instructions for utilizing fluorescence polarization to identify the enzyme activity; a receptacle containing a reporter molecule; and, a receptacle containing a binding protein.

Reference is now made in detail to the preferred embodiments of the invention, examples of which are illustrated in the accompanying drawings.

### BRIEF DESCRIPTION OF THE DRAWINGS

FIG. 1 is a cartoon illustrating the direct detection of phosphorylated amino acids using fluorescence polarization.

FIG. 2 is a cartoon illustrating competitive detection of phosphorylated amino acids using fluorescence polarization.

FIG. 3 is a cartoon illustrating the detection of amino acid dephosphorylation.

FIG. 4 is a line graph indicating that as the antibody binds to a fluorescently labeled phosphopeptide, the polarization value increases.

FIG. 5 is a line graph showing that antibody bound to a fluorescently labeled phosphopeptide can be competed off with an unlabeled phosphopeptide.

FIG. 6 is a bar graph demonstrating that PKC activity can be measured by using competitive fluorescence polarization.

FIG. 7 is a line graph showing the quantitative measurement of kinase activity.

FIG. 8 is a line graph demonstrating the binding measurements between different antibodies and fluorescein labeled peptides containing phosphotyrosine.

FIG. 9 is a line graph illustrating that a competition assay can be used quantitatively to measure the amount of phosphorylated amino acid in a mixture.

FIG. 10 is a scatter graph showing a kinase activity measurement using competitive fluorescence polarization.

FIG. 11 is a scatter graph showing the autophosphorylation of an EGF receptor.

FIG. 12 is a scatter graph showing a tyrosine kinase assay performed with and without the inhibitor EDTA.

FIG. 13 is a scatter graph illustrating a high polarization mixture containing an antibody bound to a fluorescein labeled phosphopeptide.

### DETAILED DESCRIPTION OF THE INVENTION

This invention provides a simple, homogeneous, nonradioactive method for detecting phosphorylated amino acids which allows for quantitative measurement of protein kinase and phosphatase activity and for identification of enzyme inhibitors. The method is based on the discriminate recognition of phosphorylated versus unphosphorylated amino acids by proteins such as antibodies or SH2 domains. It is also based on the ability of fluorescence polarization measurements which distinguish between a small fluorescently labeled molecule and the same molecule when it is bound to a large antibody or other protein. Our method depicts a change in fluorescence polarization signal with a change in concentration of phosphorylated amino acid. The change in concentration can be produced by: 1) a protein kinase adding phosphate groups, 2) a phosphatase removing phosphate groups, or 3) utilizing different amounts of an extract which contains phosphorylated amino acids.

Detection of modified amino acids is performed using either a direct assay or a competition assay. In the direct assay, a small fluorescently labeled substrate is phosphorylated and then bound to an antibody. As more of the substrate in the reaction is phosphorylated, then more of it binds to the antibody, and the higher the polarization rises. The increase in the fluorescence polarization signal is directly proportional to the amount of phosphorylated peptide, which in turn is directly proportional to the amount of kinase activity present.

In a preferred embodiment, a competition assay is performed. The kinase reaction uses standard reagents with no labels for fluorescence or capture and no limitations on substrate size or concentration. The synthesis of the phosphorylated peptide during the reaction is detected by adding a high polarization complex to the reaction. The complex comprises a fluorescently labeled phosphopeptide bound to an antibody. As the reaction mixture and the high polarization complex are combined, the antibody will reach equilibrium between the phosphorylated amino acids from both sources. As the antibody is released from the fluorescently labeled phosphopeptide, the polarization value goes down in proportion to the amount of phosphorylated amino acids made by the kinase reaction. In this case, the fluorescence polarization signal is inversely proportional to the amount of kinase activity present.

In another preferred embodiment, our method can be used to measure the removal of phosphate groups from amino acids. The phosphatase assay is a direct assay: this means that a fluorescently labeled peptide is treated with the phosphatase; an antibody is then added which binds to the peptide; if the phosphate group is still present, the polarization value rises; if the phosphate group is removed, then the polarization remains low. This assay can also be monitored using a simple complete assay where all of the components, including the high polarization mixture, are added to a test tube except the kinase. The kinase may then be added and the starting high polarization decreases as the phosphatase removes phosphate from the peptide.

### Definitions

Binding molecule—A molecule that has an affinity for another specific molecule. In the preferred embodiments, a binding molecule can be a protein or, more specifically, an antibody specific for a phosphorylated amino acid.

Peptide—a molecule made up of 2 or more amino acids. The amino acids may be naturally occurring or synthetic.

Reporter complex—a fluorescence-emitting compound attached to a phosphorylated amino acid bound to a binding molecule.

Reporter molecules—Chemical (organic or inorganic) molecules or groups capable of being detected and quantitated in the laboratory. Reporter molecules include fluorescence-emitting molecules (which include fluoresceins, rhodamines, pyrenes, lucifer yellow, BODIPY®, malachite green, coumarins, dansyl derivatives, mansyl derivatives, dabsyl drivatives, NBD flouride, stillbenes, anthrocenes, acridines, rosamines, TNS chloride, ATTO-TAG™, Lissamine® derivatives, eosins, naphthalene derivatives, ethidium bromide derivatives, thiazole orange derivatives, ethenoadenosines, CyDyes™, aconitine, Oregon Green, Cascade Blue, and other fluorescent molecules). In the preferred embodiments, the reporter molecule comprises a fluorescence-emitting peptide molecule.

### EXAMPLES

### Example 1

Fluorescein Amine Labeling Protocol for Labeling Peptides

The pp60c-src C-terminal phosphoregulatory peptide (BIOMOL; Plymouth Meeting, Pa.) was fluorescently labeled according to the instructions included with the fluorescein amine labeling kit (PanVera Corporation; Madison, Wis.). Briefly, 50 mg of the peptide was labeled at 37° C. for one hour in a 50 mL reaction containing 5 mL 10×coupling buffer (1 M KPO4, pH 7.0) and 5 mL 20 mM fluorescein. The reaction was then quenched with 5 mL 1 M Tris-HCl (pH 8.0) and incubated at room temperature for 30 minutes. Fluorescein-labeled products were then separated by thin layer chromatography. Using silica TLC plates, 2 mL of the reaction products were spotted onto the origin. The products were then separated for 6 hours using 4:1:1 n-butanol:acetic acid:water (v/v) as the solvent. The plate was then dried and photographed, and fluorescein-labeled peptides were scraped off the plate and eluted into 200 mL 50 mM Tris-HCl (pH 8.0).

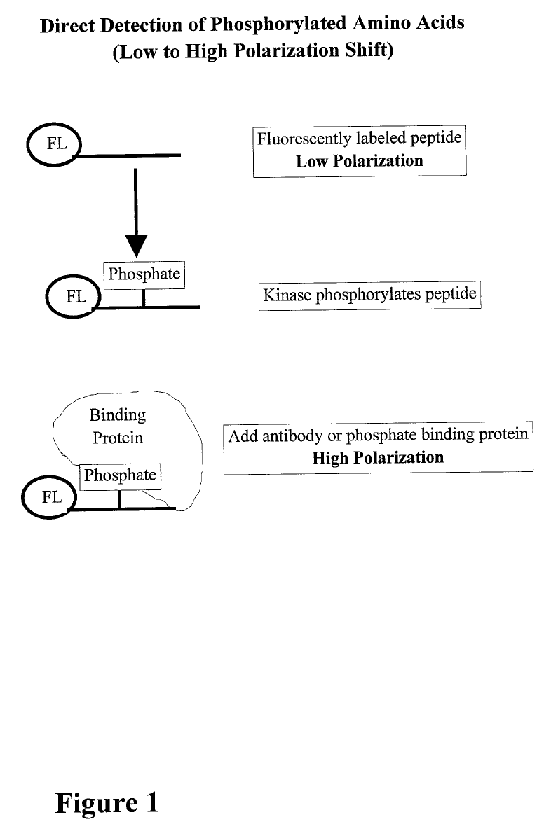

As shown in FIG. 1, a short peptide is fluorescently labeled and used as a substrate in the kinase reaction. The kinase adds a phosphate group to the amino acid and then a protein which binds to the phosphate is added to the reaction. The starting fluorescent peptide has a low polarization value and when the protein binds to it, it has a high polarization value. This is a good assay for determining whether or not a peptide is a substrate for a kinase.

### Example 2

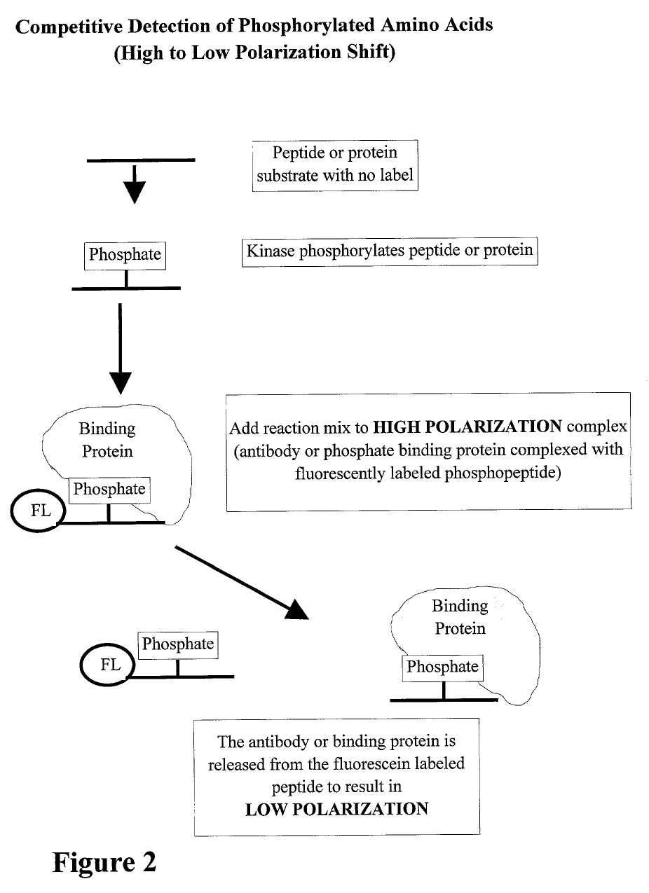

In FIG. 2, the substrate for the kinase can be any peptide or protein and size is not a limitation. Also, there is no limitation on the concentration of the peptide or protein. Once the kinase reaction has occurred, a high polarization complex is the added to the reaction. This complex contains a fluorescently labeled phosphorylated peptide bound to protein. When the reaction and high polarization complex are mixed, the phosphorylated amino acids in the reaction compete for binding to the proteins in a high polarization mix. As the binding proteins are released from the fluorescently labeled peptide, the polarization value of the peptide goes down. In this case the shift in polarization is from a high polarization complex to a low polarization free phosphopeptide. In a simplified version of this assay, both the kinase reaction and the high polarization complex are mixed together at the beginning of the reaction. As the reaction proceeds, the polarization value goes down.

### Example 3

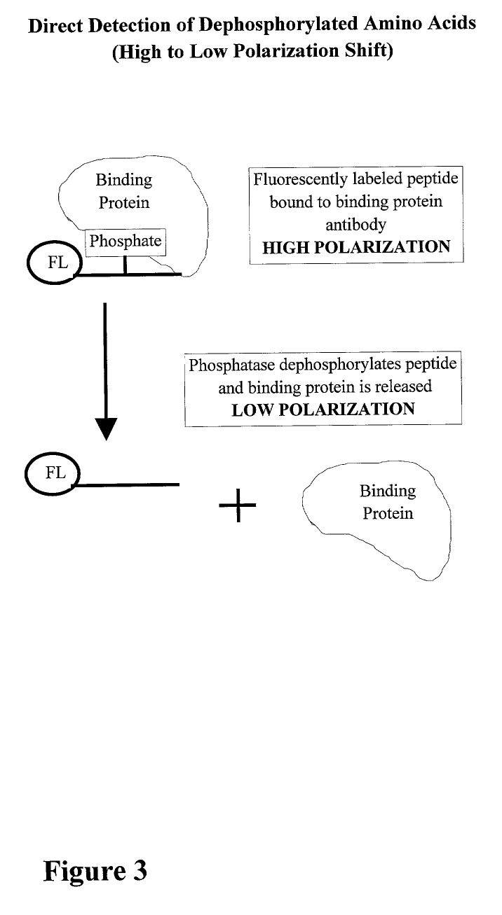

In FIG. 3, the reaction begins with a high polarization mixture, which is a binding protein attached to a fluorescently labeled phosphopeptide. A phosphatase, an enzyme that removes phosphate groups from other molecules, is added to the reaction and the polarization value goes down. Phosphatase enzymes are important in cellular regulation because they perform the opposite role of kinases.

### Example 4

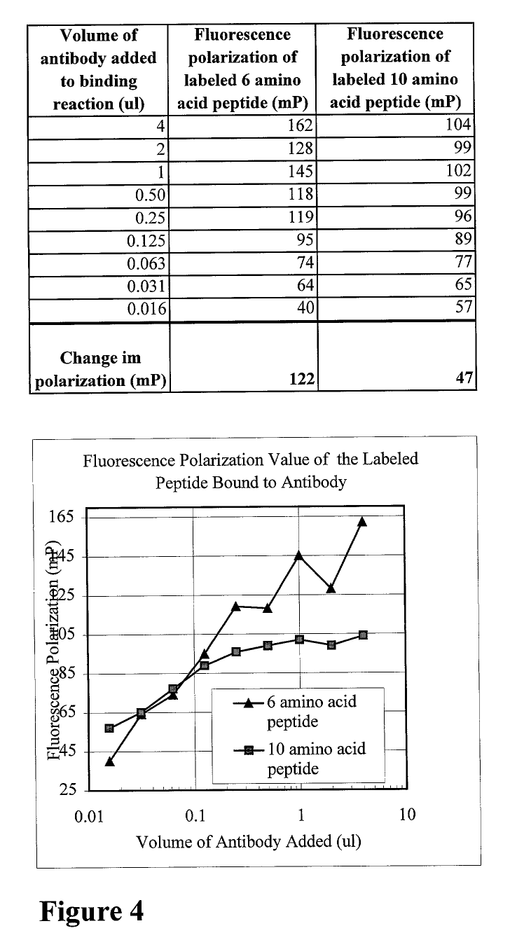

The shift in polarization (FIG. 4) of a fluorescein-labeled 11 mer peptide (F-RRVTpSARRS) and fluorescein-labeled 6 mer (F-pSAARRS) upon addition of antiphosphopeptide antibody (anti-GFAP-P, MBL clone YC10) was measured. The anti-GFAP-P was serially diluted in 100 mM potassium phosphate, 100 μg/ml bovine gamma globulin (BGG) in two sets from 4 μl to 0.016 μl per reaction. The fluorescein-labeled 6 mer and 11 mer were added to sets 1 and 2, respectively to a final concentration of approximately 0.5 nM. Polarization values were measured after a 30 minute incubation and plotted versus the Log of fluorescein-peptide concentration.

FIG. 4 shows that peptide is a substrate for PKC, and the phosphorylated peptide contains phosphoserine. It also shows that by decreasing the size of the peptide, it is possible to get a higher shift in polarization. The monoclonal antibody was supplied by MBL International Corporation, Watertown, Mass. and it is very specific for this peptide. The phosphoserine and phosphothreonine are small molecules compared to phosphotyrosine. To make antibodies against these phosphopeptides, the phosphotyrosine is large enough that it can constitute an epitope for antibody recognition almost by itself, with little recognition of the amino acids surrounding phosphate containing amino acids. In contrast, an antibody prepared against a phosphoserine containing peptide will also recognize the amino acids adjacent to the phosphorylated amino acids. This means that antibodies used in the kinase reactions are very specific to individual peptides.

### Example 5

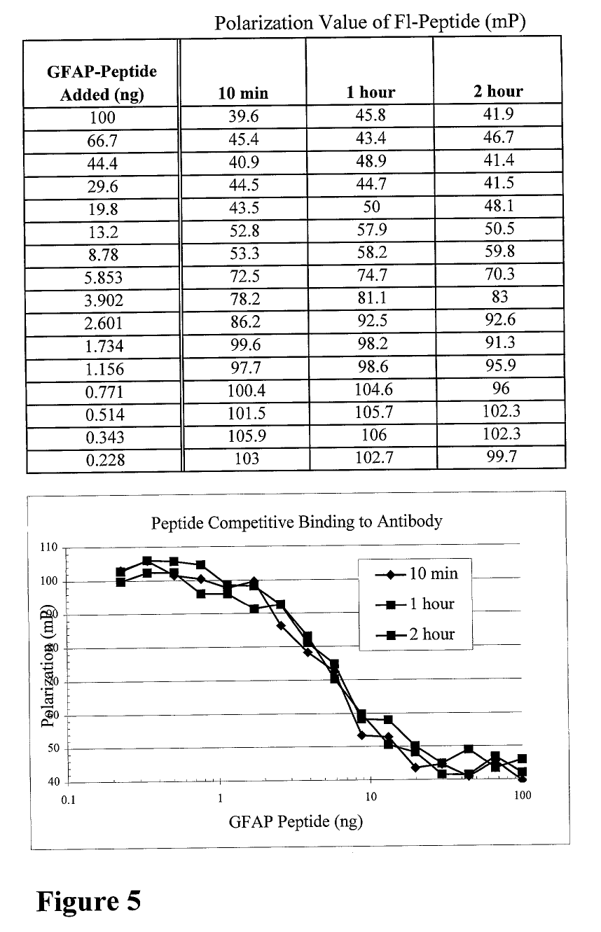

An experiment (FIG. 5) was performed to measure the ability of the unlabeled phosphopeptide (GFAP-P) to compete with the fluorescein-labeled GFAP-P (GFAP-P-F) for binding to Anti-GFAP-P. GFAP-P was serially diluted in 16 tubes from 100 ng to 0.2 ng in 100 μL final volumes. Then 50 pg GFAP-P-F and 1 μl Anti-GFAP-P were added to each reaction tube. Fluorescence polarization was measured at 10 minutes, 1, and 2 hours in all tubes. Data was plotted as polarization versus Log peptide concentration.

In FIG. 5, the peptide contains a phosphoserine, and when bound to an antibody has a high polarization value (100 mP). As increasing amounts of the unlabeled peptide are added, the polarization value decreases. The decrease in polarization is directly proportional to the amount of competitor peptide added.

### Example 6

The ability of three PKC isozymes (Beta2, Delta, and Epsilon) to phosphorylate the GFAP to GFAP-P was measured as well as a mock reaction with no enzyme added. Kinase reactions were performed containing PKC or buffer, 63 μg/ml GFAP peptide, 63 μM ATP, 30 mM HEPES, 6 mM MgCl2, 63 μM CaCl2, phosphatidyl serine (100 μg/ml) and diacylglycerol (100 μg/ml). Reactions were incubated at 30° C. for 30 minutes, then placed on ice. The amount of phosphopeptide produced was measured by competition with a anti-GFAP-P and GFAP-P-F. In the competition experiment 50 μl of each kinase reaction, 1 μl of anti-GFAP, and 50 pg F-GFAP-P, were added to 1 ml PBS. A negative control with no reaction added and positive control with no reaction and no antibody added were also performed. Polarization values were measured. The presence of GFAP-P, produced in the kinase reactions, was demonstrated by the reduced polarization values in the tubes containing the kinase reactions. The tubes containing no reaction or a mock reaction showed no drop in polarization, while the tube containing no antibody showed the polarization value of the free GFAP-P-F.

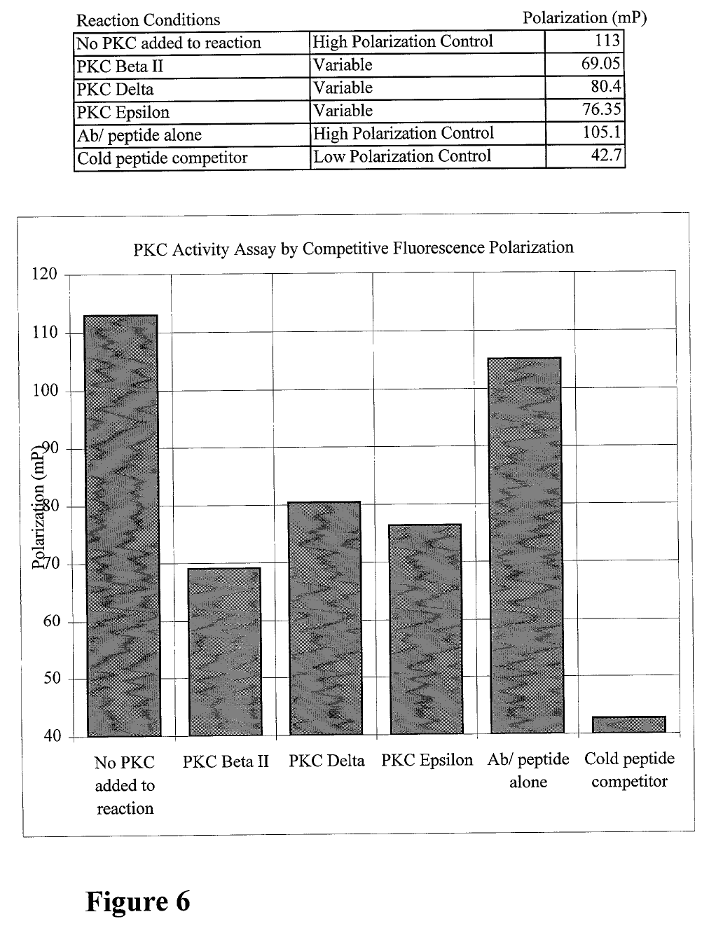

In FIG. 6, the duplicate reactions were set up with one reaction being spiked with radioactive ATP and the other reaction performed by fluorescence polarization. In the radioactive assays the reaction which had no enzyme added did not show incorporation of the radioactive nucleotide. Three of the different PKC isoforms were tested in these reactions. All three of the enzymes showed activity in the radioactive assay. To test the enzyme activity using fluorescence polarization, the reactions were allowed to proceed and then the high polarization complex of antibody and phosphopeptide were added to the reactions. The phosphorylated amino acids which were made by the kinase reactions competitively bound to the antibody, thereby releasing the fluorescein labeled phosphopeptide. This caused a decrease in the fluorescence polarization value. When no PKC was added the polarization value stayed high. When the antibody and the fluorescent peptide were added together the polarization also remained high. When phosphopeptide was added to the high polarization complex, it competed off the antibody and therefore explains the lower the polarization value. All three of the PKC isoforms showed intermediate values in polarization, demonstrating that they had produced intermediate levels of phosphorylated amino acids.

The radioactive data for FIG. 6 showed that the PKC beta II enzymes phosphorylated 0.82% of the substrate, PKC delta phosphorylated 3.27% of the substrate, and PKC epsilon phosphorylated 1.47% of the substrate. The background level where no PKC was added to the reaction showed a level of 0.05% of the substrate being phosphorylated. The method for the radioactive measurement follows:

Radioactive Kinase Assay:

The PKC activity assay was performed by a traditional radioactive protocol to compare with a competitive fluorescence polarization assay. The radioactive (³²P) was performed according to the standard protocol with the following alterations:

All final concentrations of reagents were identical (although 100 μl reactions were utilized, not 60 μl) with the following exceptions:

Final ATP concentration of 10 μM (rather that 100 μM)

No EGTA was added

Final concentration of 100 μM CaCl₂

Peptide substrate utilized was the GFAP peptide at 100 μg/ml final.

Mix was prepared without ³²P-ATP and tubes were aliquoted for FP assay. ³²P-ATP was then added to the reaction mix and tubes aliquoted for the hot assay. Enzyme preparation is then added to each tube.

PKC Activity Assay Protocol

Purpose/Discussion

The purpose of this protocol is to provide the reader with the conditions that we use to determine the activity and phospholipid dependence of Protein Kinase C ε. This protein is purified to near homogeneity and may behave differently from crude preparations. The calculated molecular weight of the protein is 83.5 kDa. Apparent molecular weights of 89-96 kDa have been reported in the literature, and the protein may run as a doublet (1). PKC ε is classified as a novel protein kinase C because it does not show calcium dependence, and is activated in vitro by phorbol esters. With the assay described in this protocol, we typically see a two-fold phosphatidylserine stimulation of activity. There are many factors that can affect the phospholipid dependence of this enzyme and it will display different degrees of dependency for different substrates. The quality of the lipids used is very important and can affect the lipid dependency dramatically. The reaction conditions given in this protocol and the enzyme concentration ranges used give linear reaction kinetics over at least ten minutes.

Safety Precautions

Normal precautions exercised in handling laboratory reagents should be followed.

Activity Assay Protocol

Reaction Mix Composition

| Reaction Mix | Stocks to Use |
|---|---|
| 20 mM HEPES, pH 7.4 | 0.5M HEPES, pH 7 |
| 0.1 mM EGTA | 1 mM EGTA |
| 10 mM MgCl₂ | 100 mM MgCl₂ |
| 100 μg/ml ε substrate peptide | 1 mg/ml PKC Epsilon (ε) substrate |
| | peptide |
| | (ERMRPRKRQGSVRRRV) |
| 100 μM ATP | 10 mM ATP |
| 200 μg/ml Phosphatidylserine | 10 mg/ml PS (Sigma - P6641) |
| 20 μg/ml diacylglycerol | 2 mg/ml DAG (Sigma - D0138) |
| 0.03% Triton X-100 | from Lipid Mix Resuspension |
| | Buffer |
| Trace (γ ³²P)ATP | — |

Lipid Mix Preparation

20 μg of Phosphatidylserine (1.2 μl of 10 mg/ml PS Stock) and 2 μg of diacylglycerol (0.6 μl of 2 mg/ml DAG Stock) are needed per reaction. Determine the total amount of each needed for the number of reactions one is running and make up 10% more lipid mix than required to account for pipetting loss.

Using a Hamilton syringe (remember to clean out with methanol), transfer the required volume of each lipid stock to a 12×75 mm glass test tube. Thoroughly dry down the chloroform with a nitrogen stream and gentle rotation of the tube. Resuspend the dried mixture with 10 μl buffer per reaction. Resuspension buffer is 10 mM HEPES, pH 7.4/0.3% Triton X-100. Vortex into solution/suspension. This will take at least two minutes of vortexing. Place the lipid mix in a 40° C. water bath for 5 minutes prior to adding to the reaction mix.

Procedure

1). Make up the reaction mix. All assays should be done in triplicate. Two blanks (reaction mix with no enzyme added) should be included and one tube for determining the total cpm's in a reaction.

| Volume | Reagent |
|---|---|
| 2.4 μl | 0.5M HEPES, pH 7.4 |
| 6 μl | 100 mM MgCl₂ |
| 6 μl | 1 mM EGTA (100 mM stock diluted 1/100) |
| 6 μl | 1 mg/ml PKC ε peptide stock stored at −20° C. |
| 0.6 μl | 10 mM ATP stock stored at −20° C. |
| 6 μl | Lipid mix |
| 0.1 μl | (γ³²P) ATP (add more if isostope is more than one |
| | week old) |
| 32.9 μl | distilled H₂O |
| 60 μl | TOTAL |

2). Dispense 60 μl of mix into each tube and place tubes at 30° C.

3). Dilute the protein to be assayed to between 20 and 50 ng/μl.

Dilution Buffer

10 mM Tris-HCl, pH 7.5
5 mM DTT
0.01% Triton X-100

Since it is dificult to make accurate dilutions pipetting small volumes (<5 μl), we recommend using at least 5 μl of enzyme in the dilution. Example: 1/100dilution—add 5 μl of enzyme to 495 μl of dilution buffer.

4). Add 2 μl of diluted enzyme to each tube at 20 second intervals. Blanks have no enzyme added.

5). Stop reactions at 10 minutes by adding 6 μl 5% phosphoric acid.

6). Incubate on ice for 5 minutes.

7). Spot 5 μl of reaction mix on to two phosphocellulose membranes. These are the “Totals”.

8). Transfer 55 μl from each tube to phosphocellulose membranes. Allow to dry. Wash the membranes three times with 5 ml 0.5% phosphoric acid per filter in a 400 ml beaker.

9). Transfer membranes to scintillation vials and count (drying is not necessary).

Unit Definition

One unit is defined as the amount of enzyme necessary to transfer 1 nmol of phosphate to the PKC Epsilon (ε) substrate peptide (supplied by PanVera, Inc.) in 1 minute at 30° C.

Activity Calculation

$$units / μ l = \frac{( cpm sample - blank ) \times dilution factor}{( cpm for 5 μ l of rxn mix ) \times 33.4}$$

NOTE: The 33.4 in the denominator is the result of: 5 nmol ATP

50 μl rxn mix×1.67 μl enzyme added×10 minutes

5 μl counted

Phospholipid Dependence

Phospholipid dependence is shown by following the procedure above and simultaneously running reactions to which no lipid has been added. The reaction volume is made up by adding lipid resuspension buffer in section 3.3 instead of lipid. The reactions without lipid are processed identically to those with lipid.

Below are the values that we obtained with the current lot of our enzyme.

| Reaction Conditions | Specific Activity/% Activity |
|---|---|
| | PKC ε lot #6747 |
| PS/DAG | 1250/100% |
| No Lipid Activators | 743/59% |

The foregoing is considered as illustrative only of the principles of the invention. Further, since numerous modifications and changes will readily occur to those skilled in the art, it is not desired to limit the invention to the exact construction and operation shown and described. Therefore, all suitable modifications and equivalents fall within the scope of the invention.

### Example 7

A “complete” and “mock” (no enzyme) PKC B2 reactions were performed. The reactions contained PKC Beta2 or buffer, 63 μg/ml GFAP peptide, 63 μM ATP, 30 mM HEPES, 6 mM MgCl2, 63 μM CaCl2, phosphatidyl serine (100 μg/ml) and diacylglycerol (100 μg/ml). Reactions were incubated at 30° C. for 30 minutes, then placed on ice. The reactions were serially diluted in BGG/P buffer. 10 μl of a complex of F-GFAP-P and antibody was added to each tube in both sets (enzyme and no enzyme) and then the polarization was measured. The mock reaction had no effect on the complex, while the polarization drop in the complete reaction was dose-dependent on the amount of reaction added.

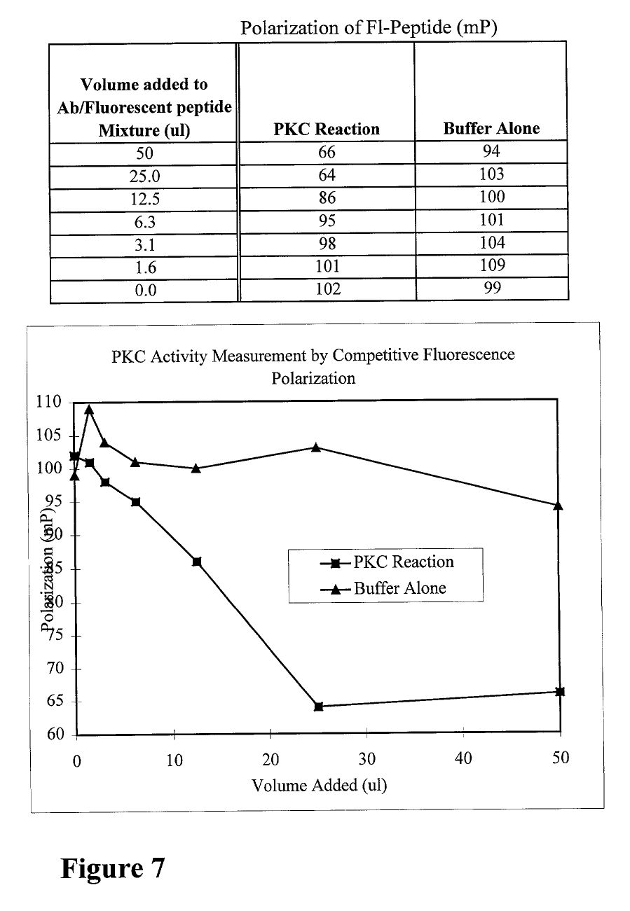

In FIG. 7, a single kinase reaction was set up and allowed to proceed in phosphorylated the peptide substrate. Different amounts of the kinase reaction were then ended to tubes which contains the high polarization antibody—peptide mixture. As FIG. 7 shows, larger amounts of the reaction mixture caused a lower polarization value. As a control, the buffer alone was substituted for the kinase reaction mixture. This did not show any significant change in the polarization value.

FIGS. 1-7 dealt with kinases which add phosphate groups to serine and threonine amino acids and with antibodies that binds these phosphopeptides. As shown in FIG. 7, some of the antibodies bind the phosphopeptide more tightly and cause a higher shift in polarization. The higher shift in polarization produces a better assay, because it is easier to measure the decreasing polarization using a competition assay. FIG. 4 shows that the different length of peptide can affect the polarization value. This figure shows that different antibodies can also have a significant effect. Therefore, to optimize the assay is necessary to find the best combination of antibody and peptide.

### Example 8

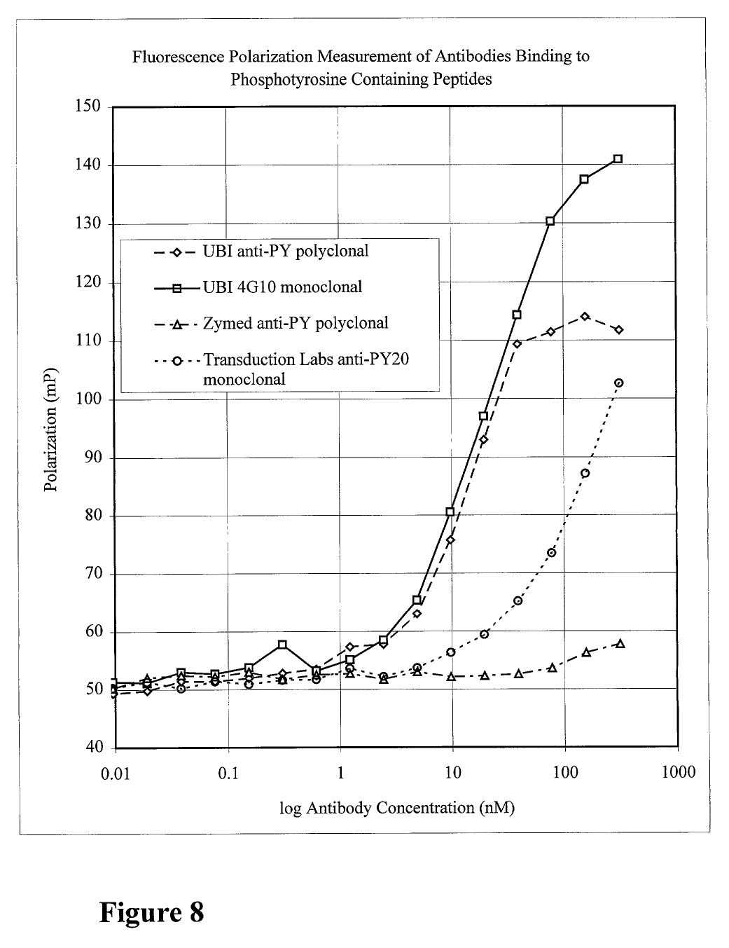

Fluorescence polarization changes (FIG. 8) caused by four commercially available anti-phosphotyrosine antibodies were measured using 7.35 nM of the pp60c-src C-terminal phosphoregulatory peptide (BIOMOL; Plymouth Meeting, Pa.) as the fluorescent tracer. Each serial dilution was done in final volume of 100 mL in Beacon™-grade phosphate-buffered saline (PanVera Corporation; Madison, Wis.) which contains 1.2 mM monobasic potassium phosphate, 8.1 mM dibasic sodium phosphate, 2.7 mM KCl, 138 mM NaCl, 0.02% sodium azide (pH 7.5). Each sample was read at 25° C. after a 15 minute equilibration. The antibodies were from Upstate Biotechnology (Lake Placid, N.Y.), Zymed (South San Francisco, Calif.), and Transduction Labs (Lexington, Ky.).

FIG. 8 demonstrates that different antibodies against phosphotyrosine have different binding affinities and can cause different shifts in polarization values. The optimal antibodies give the highest shift at the lowest concentration, and therefore the most sensitive assays.

### Example 9

A peptide competition standard curve was generated in final volume of 100 mL Beacon™ -grade phosphate-buffered saline (PanVera Corporation; Madison, Wis.) which contains 1.2 mM monobasic potassium phosphate, 8.1 mM dibasic sodium phosphate, 2.7 mM KCl, 138 mM NaCl, 0.02% sodium azide (pH 7.5). Each tube also contained 20 nM 4G10 anti-phosphotyrosine monoclonal antibody (Upstate Biotechnology; Lake Placid, N.Y.), 10 nM fluorescent pp60c-src C-terminal phosphoregulatory peptide (BIOMOL; Plymouth Meeting, Pa.), and serially diluted, non-phosphopeptide (same sequence as the pp60c-src C-terminal phosphoregulatory peptide) as the competitor peptide (BIOMOL; Plymouth Meeting, Pa.). Each tube was analyzed at 25° C. after a 15 minute equilibration.

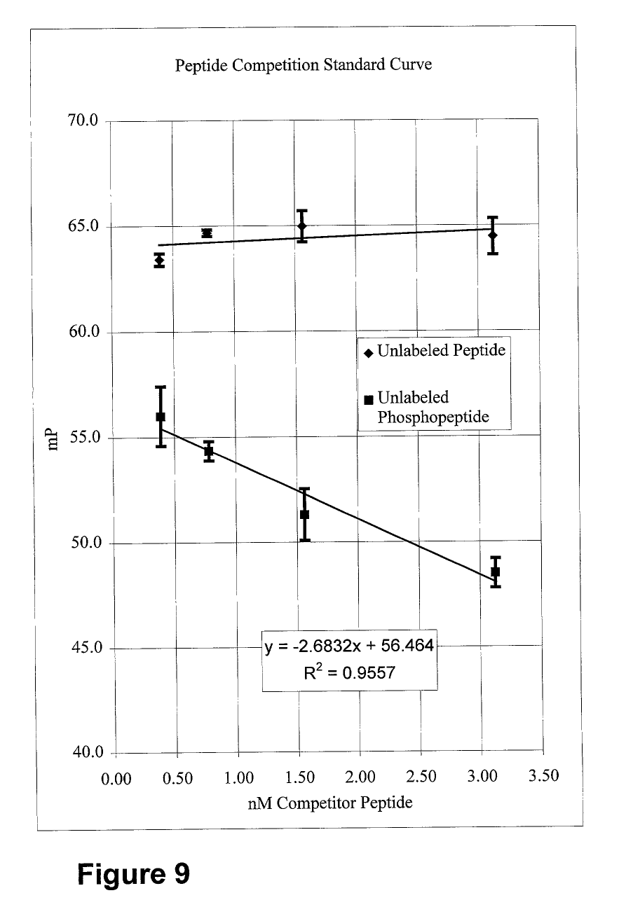

As shown in FIG. 9, even when the total shift in polarization is less than 10 mP, it is possible to detect phosphorylated amino acids. This assay could be optimized to give a much larger dynamic range of detection.

### Example 10

A change in fluorescence polarization was detected using varying amounts the epidermal growth factor (EGF) receptor tyrosine kinase. The kinase was purified according to the methods of Weber et al. (1984) J. Biol. Chem. 259:14631-6 and was obtained from Dr. Paul Bertics (University of Wisconsin-Madison). Each reaction was run in a final volume of 100 mL under the following conditions: 20 mM Hepes (pH 7.4), 2 mM MgCl₂, 5 mM MnCl₂, 50 mM Na₃VO₄, 50 mM ATP, 1 mM unphosphorylated pp60c-src C-terminal regulatory peptide (BIOMOL; Plymouth Meeting, Pa.), 20 nM 4G10 anti-phosphotyrosine monoclonal antibody (Upstate Biotechnology; Lake Placid, N.Y.), and 10 nM fluorescent pp60c-src C-terminal phosphoregulatory peptide (BIOMOL; Plymouth Meeting, Pa.).

The change in polarization was measured every 10 seconds (after the addition of the kinase) in Kinetic Mode on a Beacon™ 2000 instrument (PanVera Corporation; Madison, Wis.) running at 30° C.

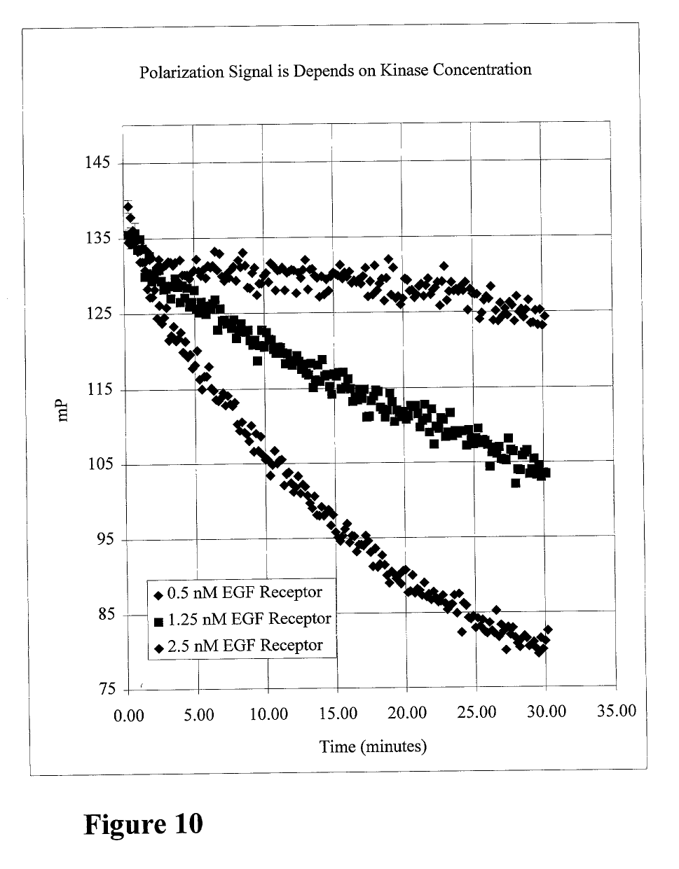

In FIG. 10, three reactions were performed using increasing amounts of the EGF receptor, a tyrosine kinase. In each tube, the reaction and the detection were performed simultaneously. All the kinase reaction components and the high polarization mixture were added to the tube, the reaction started, and a polarization value was measured every few seconds for 30 minutes. As the kinase reaction proceeded, the polarization value went down. The polarization value was lower when higher levels of the kinase were used. As was previously shown, these assays can be performed as end-point assays. The results shown in this figure shows that this is the simplest way to perform the assay as long as the presence of the antibody and the fluorescently labeled peptide do not interfere with the kinase reaction.

### Example 11

Autophosphorylation of the EGF receptor can also be detected using fluorescence polarization. The 100 mL reaction was assayed under the following conditions: ˜12.5 nM EGF receptor, 20 mM Hepes (pH 7.4), 2 mM MgCl2, 5 mM MnCl2, 50 mM Na3VO4, 50 mM ATP, 20 nM 4G10 anti-phosphotyrosine monoclonal antibody (Upstate Biotechnology; Lake Placid, N.Y.), and 10 nM fluorescent pp60c-src C-terminal phosphoregulatory peptide (BIOMOL; Plymouth Meeting, Pa.). The change in polarization was measured every 10 seconds (after the addition of the kinase) in Kinetic Mode on a Beacon™ 2000 instrument (PanVera Corporation; Madison, Wis.) running at 30° C.

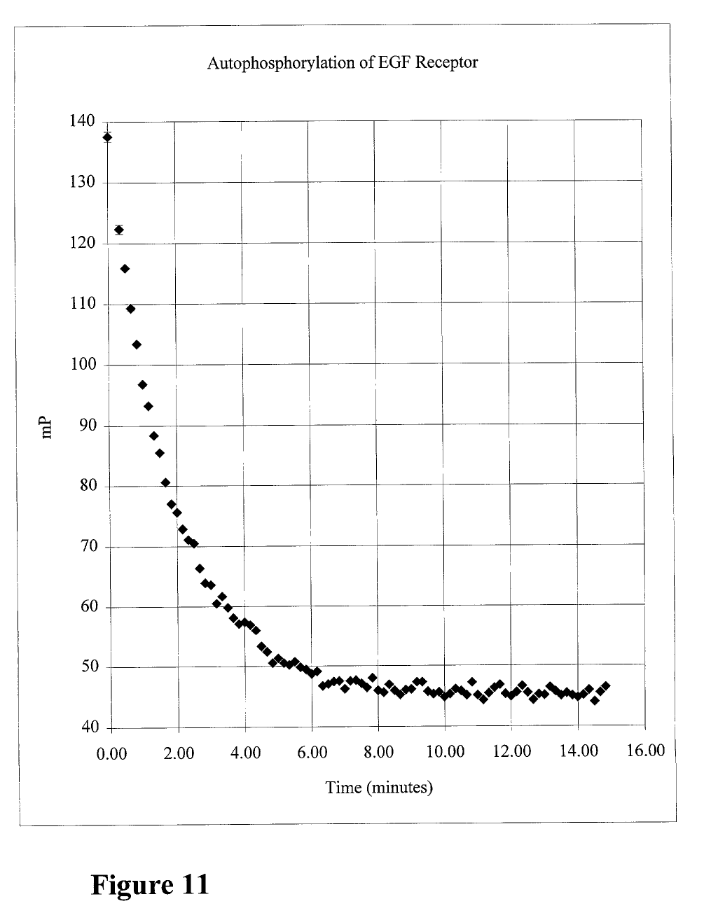

In most kinase reactions, the kinase adds the phosphate group to another molecule such as a peptide or protein. However, it is possible that the kinase could add the phosphate group to itself, which is called autophosphorylation. As shown in FIG. 11, no peptide substrate was added to the reaction and the production of phosphotyrosine was measured using the competitive fluorescence polarization assay. A previous control experiment showed that the antibody was not a significant substrate for this kinase (data not shown).

### Example 12

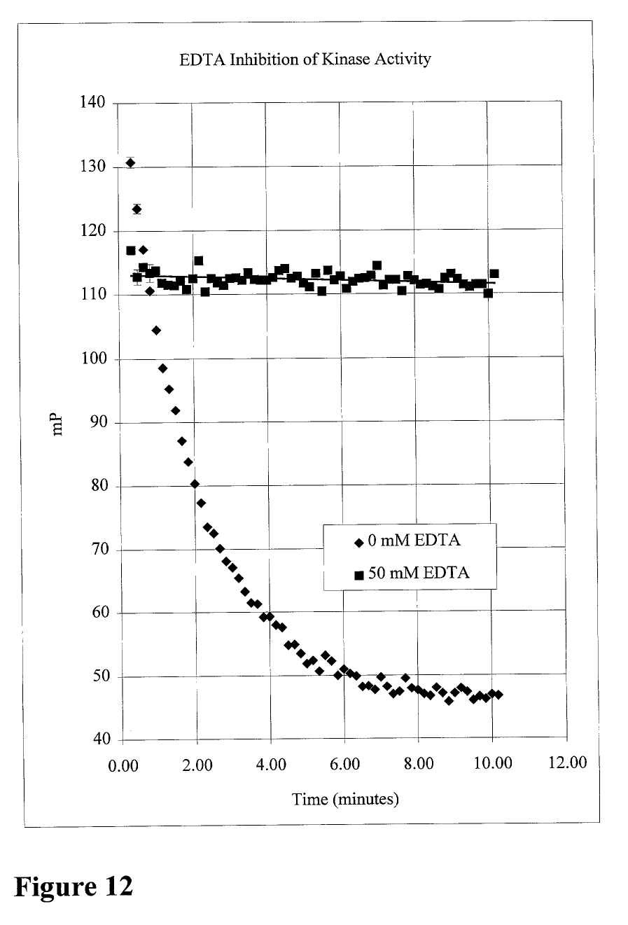

FIG. 12. EDTA inhibits the EGF receptor kinase activity. The 100 mL reactions (with or without 50 mM Beacon™-grade EDTA (PanVera Corporation; Madison, Wis.) were assayed under the following conditions: ˜12.5 nM EGF receptor, 20 mM Hepes (pH 7.4), 2 mM MgCl2, 5 mM MnCl2, 50 mM Na3VO4, 50 mM ATP, 20 nM 4G10 anti-phosphotyrosine monoclonal antibody (Upstate Biotechnology; Lake Placid, N.Y.), and 10 nM fluorescent pp60c-src C-terminal phosphoregulatory peptide (BIOMOL; Plymouth Meeting, Pa.). The change in polarization was measured every 10 seconds (after the addition of the kinase) in Kinetic Mode on a Beacon™ 2000 instrument (PanVera Corporation; Madison, Wis.) running at 30° C.

As one would expect (FIG. 12), the polarization value did not change when the inhibitor was present, but changed significantly when it was not. This demonstrates that the assay could be used to screen for selective inhibitors of kinase activity, especially in a high throughput screen format. These inhibitors could chelate the metal ions like EDTA, or bind to the active site of the enzyme, or bind to the ATP binding site on the kinase.

### Example 13

Fluorescence polarization can also be used to measure phosphatase activity. The conditions of each 100 mL assay were: 0.5 U T-cell protein tyrosine phosphatase (New England Biolabs; Beverly, Mass.), 25 mM imidazole, 50 mM NaCl, 2.5 mM Na₂EDTA, 5 mM DTT, 100 mg/mL BSA (pH 7.0), 5 nM fluorescent pp60c-src C-terminal phosphoregulatory peptide (BIOMOL; Plymouth Meeting, Pa.), 20 nM 4G10 anti-phosphotyrosine monoclonal antibody (Upstate Biotechnology; Lake Placid, N.Y.). One assay received 1.0 mM of the phosphatase inhibitor Na₃VO₄ while a another assay did not receive enzyme. The change in polarization was measured every 10 seconds (after the addition of the phosphatase) in Kinetic Mode on a Beacon™ 2000 instrument (PanVera Corporation; Madison, Wis.) running at 30° C.

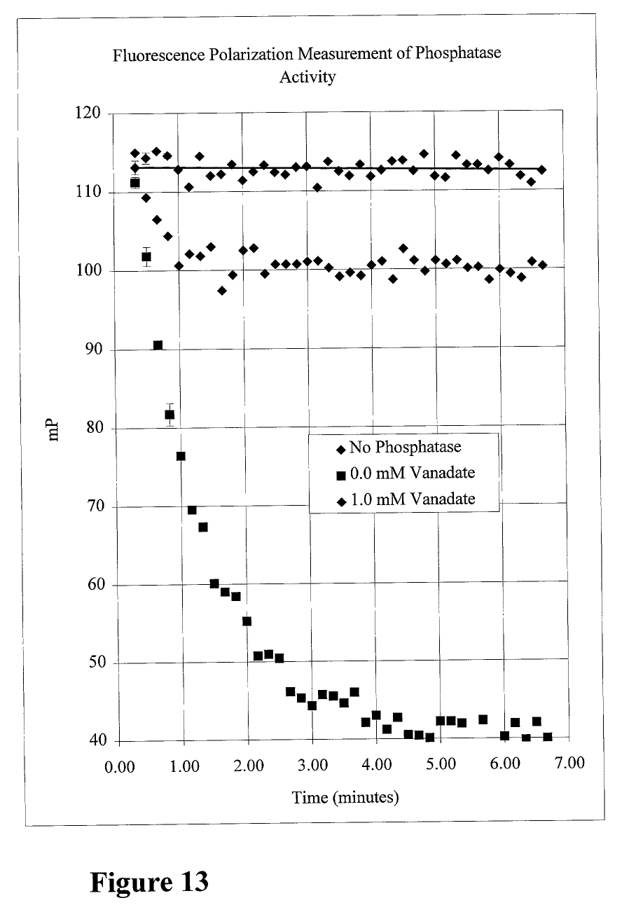

Phosphatase enzyme was added to a high polarization mixture and as it removed the phosphate from the peptide, the antibody was released, and the polarization value went down, shown in FIG. 13. Two control experiments were performed. One control showed that when no phosphatase was added, the polarization value remained constant. The second control showed that when a phosphatase inhibitor, vanadate, was added to the reaction, the polarization value also remained constant. This assay was performed with all of the reaction and detection components present in a test tube. However, this reaction could also be done as an endpoint assay with aliquots of the reaction taken at incremental time points, or with different reactions started at the same time but terminated at incremental time points.

We claim:

1. A method for measuring the presence of a phosphorylated amino acid of a compound by competition, comprising:
a) measuring the fluorescence polarization of a reporter complex comprising a first phosphorylated amino acid bound to a binding molecule;
b) adding a substance containing a second phosphorylated amino acid to compete for the binding molecule;
c) incubating the solution;
d) measuring the fluorescence polarization of the solution during step c); and,
e) comparing the fluorescence polarization measurements of step a) with step d).

2. The method of claim 1 wherein the compound is a peptide.

3. The method of claim 1 wherein the compound is a protein.

4. The method of claim 1 wherein the binding molecule comprises a binding protein specific for a phosphorylated amino acid.

5. The method of claim 4 wherein the binding protein comprises an antibody specific for a phosphorylated amino acid.

6. A method for measuring enzyme activity for attaching and cleaving a phosphate with a compound by competition, comprising:
a) measuring the fluorescence polarization of a reporter molecule comprising a phosphorylated amino acid;
b) adding an enzyme and a substrate and,
c) incubating the solution;
d) measuring the fluorescence polarization of the solution during step c); and,
e) comparing the fluorescence polarization measurements of step a) with step d).

7. The method of claim 6 wherein the enzyme comprises a kinase for measuring phosphorylation.

8. The method of claim 7 wherein a binding protein is added during step c).

9. The method of claim 6 wherein the enzyme comprises a phosphatase for measuring phosphate cleavage.

10. The method of claim 9 wherein the binding protein is added during step a).

11. The method of claim 6 wherein the reporter molecule further comprises a fluorescence-emitting molecule.

12. A method for measuring enzyme activity of attaching or cleaving a phosphate and a peptide, comprising:
a) incubating a phosphorylated peptide and an enzyme in solution;
b) adding a reporter molecule to the solution;
c) measuring the fluorescence polarization of the reporter molecule in step b);
d) incubating the solution;
e) measuring the fluorescence polarization of the solution during step d); and,
f) comparing the fluorescence polarization measurements of step c) with step.

13. The method of claim 12 wherein the enzyme comprises a kinase for measuring phosphorylation such that the reporter molecule competes with the peptide when phosphorylated.

14. The method of claim 12 wherein the enzyme comprises a phosphatase for measuring phosphate cleavage such that the reporter molecule complex does not compete with the peptide if the phosphate is cleaved.

15. A kit utilizing the method of claim 1 for measuring the amount of phosphorylated molecules in a mixture, comprising:
a) instructions for utilizing fluorescence polarization to identify the amount of phosphorylated amino acids in a mixture,
b) a receptacle containing a reporter molecule; and,
c) a receptacle containing a binding protein.

16. The kit of claim 15 wherein the reporter molecular complex comprises a fluorescence-emitting peptide.

17. The kit of claim 16 wherein the binding protein comprises an antibody specific for phosphorylated amino acids.

18. The kit of claim 17 wherein the enzyme comprises kinase.

19. The kit of claim 18 wherein the enzyme comprises phosphatase.
